(19) United States
(12) Patent Application Publication
(10) Pub. No.: US 2025/0134430 A1
(43) Pub. Date: May 1, 2025

(54) METHOD AND SYSTEM FOR MENTAL STATE PERCEPTION, READABLE STORAGE MEDIUM

(71) Applicant: INSTITUTE OF AUTOMATION, CHINESE ACADEMY OF SCIENCES, Beijing (CN)

(72) Inventors: Zhenan SUN, Beijing (CN); Yiwei RU, Beijing (CN); Kunbo ZHANG, Beijing (CN); Yunlong WANG, Beijing (CN)

(73) Assignee: INSTITUTE OF AUTOMATION, CHINESE ACADEMY OF SCIENCES, Beijing (CN)

(21) Appl. No.: 19/008,644

(22) Filed: Jan. 3, 2025

Related U.S. Application Data
(63) Continuation of application No. PCT/CN2023/125006, filed on Oct. 17, 2023.

(30) Foreign Application Priority Data
Apr. 10, 2023 (CN) 202310373695.7

(51) Int. Cl. A61B 5/16; A61B 5/00; A61B 5/0205; A61B 5/024; A61B 5/08; A61B 5/11; G06T 7/00; G06T 7/20

(52) U.S. Cl. CPC A61B 5/165; A61B 5/0205; A61B 5/02416; A61B 5/11; A61B 5/163; A61B 5/167; A61B 5/725; A61B 5/726; A61B 5/7264; G06T 7/0012; G06T 7/20; A61B 5/0816; G06T 2207/20084; G06T 2207/30201

(57) ABSTRACT

Disclosed are a method and a system for mental state perception and a computer-readable storage medium. The method includes: acquiring image sequences with timestamps and millimeter-wave radar raw data with timestamps; preprocessing the image sequences and the millimeter-wave radar raw data; analyzing the head region image sequences to obtain head vibration signal features; calculating face region image sequences obtained from preprocessing by using a remote photovolumetric pulse wave recording method to obtain a first heart rate; analyzing the original millimeter-wave radar data sequence to obtain a second heart rate and a breathing rate; fusing the first heart rate, the second heart rate and the breathing rate to obtain a fused heart rate and breathing rate; performing feature extraction on facial change information by a Transformer-like network; establishing a non-contact multi-modal mental perception model for prediction to obtain a predicted result of the mental state.

### CROSS-REFERENCE TO RELATED APPLICATIONS

This application is a continuation of International Application No. PCT/CN2023/125006, filed on Oct. 17, 2023, which claims priority to Chinese Patent Application No. 202310373695.7, entitled “Method and system for mental state perception, readable storage medium”, filed on Apr. 10, 2023. All of the aforementioned applications are incorporated herein by reference in their entireties.

### TECHNICAL FIELD

The present application relates to the field of mental state perception and data processing, in particular to a method and a system for mental state perception, and a readable storage medium.

### BACKGROUND

In affective computing, the physiological signal acquisition methods for mental state perception can be mainly divided into two types, namely contact physiological signal acquisition and non-contact physiological signal acquisition. Contact physiological signal acquisition methods mainly include electroencephalograph, skin electrograph, contact heart rate meter and head-mounted eye tracker. Contact signal acquisition mainly faces the bottleneck of limited application scenarios, and contact sensing devices may make subjects introduce additional emotions during the test, which in turn affects the test results. Non-contact physiological signal acquisition methods mainly include gait acquisition, rPPG heart rate acquisition, micro-expression, etc. During the non-contact physiological signal acquisition process, measurement noise will be introduced due to motion, light, etc., so the low signal-to-noise ratio of the collected signal is the biggest challenge for non-contact physiological signal acquisition. At this stage, the signal acquisition method based on contact EEG and skin electricity can achieve a deeper mental state, but the non-contact physiological signal acquisition method still cannot obtain an accurate deeper mental state.

### SUMMARY

The present application aims to solve or improve the above technical problems.

To this end, a first aspect of this application is to provides provide a method for mental state perception.

A second aspect of the present application is to provide a system for mental state perception.

A third aspect of the present application is to provide a system for mental state perception.

A fourth aspect of the present application is to provide a computer-readable storage medium.

In order to achieve the first aspect of the present application, the technical scheme in the first aspect of the present application provides a method for mental state perception, which includes: acquiring image sequences with timestamps and millimeter-wave radar raw data with timestamps, where the image sequence comprises a plurality of non-contact physiological signals; preprocessing the image sequences and the millimeter-wave radar raw data to obtain head region image sequences, face region image sequences and an original millimeter-wave radar data sequence that are continuous in time series; analyzing the head region image sequences to obtain head vibration signal features; calculating the face region image sequences by using a remote photovolumetric pulse wave recording method to obtain a first heart rate; analyzing the original millimeter-wave radar data sequence to obtain a second heart rate and a breathing rate; fusing the first heart rate, the second heart rate and the breathing rate by using Kalman filtering to obtain a fused heart rate and a fused breathing rate; performing feature extraction on facial change information in the image sequences by a Transformer-like network to obtain facial motion temporal features; corresponding the head vibration signal features, the fused heart rate and the fused breathing rate, and the facial motion temporal features according to timestamps to obtain a corresponding physiological sequence; establishing a non-contact multi-modal mental perception model, and taking the corresponding physiological sequence as the input of the non-contact multi-modal mental perception model to predict and to obtain a predicted result of the mental state.

According to the method for mental state perception provided by the embodiment, first, the image sequences with timestamps and millimeter-wave radar original data with timestamps are acquired, the image sequence and the millimeter-wave radar raw data are preprocessed to obtain the head region image sequences, the face region image sequences and the original millimeter-wave radar data sequence that are continuous in the time series. Then, the head region image sequences is analyzed to obtain the head vibration signal features. The face region image sequences is calculated by using the remote photovolumetric pulse wave recording method so that the first heart rate can be obtained. The original millimeter-wave radar data sequence is analyzed so that the second heart rate and the breathing rate can be obtained. The first heart rate, the second heart rate and the breathing rate are combined so that they can be fused to obtain more precise heart rate and breathing rate. Feature extraction is performed on the facial change information in the image sequences by a Transformer-like network to obtain the facial motion temporal features. The head vibration signal feature, the fused heart rate, the fused breathing rate, and facial motion temporal features are corresponded according to timestamps, taking the corresponding physiological sequence as the input of the non-contact multi-modal mental perception model, for prediction by the non-contact multi-modal mental perception model, to obtain the mental state of an individual being measured. By progress in the methods for conversion, representation, enhancement of the multi-modal non-contact physiological signals and robust extraction of emotional features, get rid of contact sensing devices, expand application scenarios, and promote the fusion of cross-modal emotional data, so as to improve practical application value in many fields such as man-machine interaction, public safety and medical psychology.

In addition, the technical scheme provided by the present application may also include the following additional technical features.

In the technical scheme, the image sequence and the millimeter-wave radar raw data are preprocessed to obtain the head region image sequences, the face region image sequences and the original millimeter-wave radar data sequence that are continuous in time series, the method includes the following steps: processing the image sequence by using a head detection algorithm with a tracking algorithm to obtain head region image sequences with timestamps; processing the image sequence by using a face detection algorithm with a tracking algorithm to obtain face region image sequences with timestamps; performing filtering processing on the millimeter-wave radar raw data by using filtering algorithms and wavelet transform algorithms, to obtain the original millimeter-wave radar data with timestamps.

In this embodiment, the image sequence and the millimeter-wave radar raw data are preprocessed to obtain the head region image sequences, the face region image sequences and the original millimeter-wave radar data sequence that are continuous in the time series. Specifically, existing head detection algorithms are used to process the image sequences, the head region corresponding to each frame of image is cropped, and stored as head region image sequences with timestamp information. Existing face detection algorithms are used to process the image sequences, and the face region corresponding to each frame of the image is cropped, and stored as face region image sequences with timestamp information. The millimeter-wave radar raw data is processed by using filtering algorithms, and the results of processing are stored as the original millimeter-wave radar data sequence with timestamp information.

In the technical scheme, analyzing the head region image sequences to obtain head vibration signal features specifically includes: performing motion magnification on the head region image sequences, by using Euler motion magnification method, to obtain amplified head motions; obtaining head motion information according to inter-frame continuity of the amplified head motions and the image sequences, where the head motion information comprises one or a combination of the following: frequency, frequency distribution, frequency transformation range, amplitude, amplitude variation range, motion symmetry and motion period of the head motions in the horizontal and vertical directions; vectorizing the head motion information to obtain the head vibration signal features.

In the technical scheme, the head region image sequences is analyzed to obtain the head vibration signal features, specifically including, performing motion magnification on the head region image sequences, by using Euler motion magnification method, to obtain amplified head motions; obtaining head motion information according to inter-frame continuity of the amplified head motions and the image sequences; vectorizing the head motion information to obtain the head vibration signal features; where the head motion information comprises one or a combination of the following: frequency, frequency distribution, frequency transformation range, amplitude, amplitude variation range, motion symmetry and motion period of the head motions in the horizontal and vertical directions.

In the technical scheme, calculating the face region image sequences by using the remote photovolumetric pulse wave recording method to obtain the first heart rate specifically includes the following steps: extracting facial keypoints from the face region image sequences by using a keypoint detection algorithm; extracting facial skin regions according to the facial keypoints to obtain facial skin; performing facial Patch division according to the facial skin, to obtain division results; extracting BVP signals according to the division results to obtain the first heart rate.

In the technical scheme, calculating the face region image sequences by using the remote photovolumetric pulse wave recording method to obtain the first heart rate specifically includes the following steps: according to the face region image sequences, the facial keypoints are extracted by using a keypoint detection algorithm, and the facial skin regions are extracted according to the extracted keypoints (this process can avoid the interference from a complex background), and then facial Patch division (Patch division on facial skins) is performed by using the positions of the keypoints. The facial Patch division can avoid the problem of excessive noise of the measurement signal caused by uneven illumination (Patch division details), and the BVP signals are extracted, and the heart rate information of the individual being measured can be finally obtained.

In the above technical scheme, the formula of the Kalman filter is:

$${\hat{x}}_{k} = [ \begin{matrix}Heart Rate \\ Breathing Rate\end{matrix} ] ; \\ {P}_{k} = [ \begin{matrix}{Cov}_{hh} & {Cov}_{hb} \\ {Cov}_{bh} & {Cov}_{bb}\end{matrix} ] ; \\ {\hat{x}}_{k} = {F}_{k} {\hat{x}}_{k - 1} ; \\ {P}_{k} = {F}_{k} {P}_{k - 1} {F}_{k}^{T} ; \\ K = {H}_{k} {P}_{k} {{H}_{k}^{T} ( {H}_{k} {P}_{k} {H}_{k}^{T} + {R}_{k} )}^{- 1} ; \\ {{x}^{=}}_{k} = {\hat{x}}_{k} + K ( {\overline{z}}_{k} - {H}_{k} {\hat{x}}_{k} ) ; \\ {{P}^{=}}_{k} = {P}_{k} - {KH}_{k} {P}_{k} ;$$

where, \hat{x}ₖ is a millimeter-wave radar measurement value of heart rate and breathing rate at time k, Pₖ is a covariance matrix of heart rate and breathing rate, Covₕₕ represents a covariance of the heart rate and breathing rate in \hat{x}ₖ. Fₖ is a state transition matrix from k−1 to k, Hₖ is a result of rPPG heart rate measurement at time k, Rₖ represents a variance of uncertainty in heart rate measurement, z̅ₖ is an average value of Rₖ, x̅ₖ is the fused heart rate, P̅ₖ is the fused breathing rate, and K is Kalman filtering gain.

In this technical scheme, Kalman filtering is used to fuse the first heart rate, the second heart rate and the breathing rate. Kalman filtering, based on Bayesian estimation theory and considering the covariance between rPPG and mmWave, assigns larger weights to items with small errors and smaller weights to items with large errors, so as to minimize the error of the predicted result.

In the above technical scheme, establishing the non-contact multi-modal mental perception model, and taking the corresponding physiological sequence as the input of the non-contact multi-modal mental perception model to predict and to obtain the predicted result of the mental state, specifically includes: performing normalization processing on the fused heart rate and the fused breathing rate to obtain fused features; performing feature normalization processing on the head vibration signal features to obtain head vibration features; performing concat-connection on the fused features, the head vibration features and the facial motion temporal features to obtain multi-modal features; classifying the multi-modal features by using a convolutional neural network to obtain the predicted result of the mental state.

In this technical scheme, the non-contact multi-modal mental perception model is established, and a corresponding physiological sequence is used as the input of the non-contact multi-modal mental perception model for prediction, so as to obtain the predicted result of the mental state, specifically, the fused heart rate and the fused breathing rate are normalized to obtain fused features. Temporal characteristics of the head vibration are extracted to obtain the features of head vibration. Features are extracted from the temporal information of facial motions in expression and head by using an MViT2 network, to obtain features of facial motion in expression and head. The fused features, the head vibration features, the features of facial motion in expression and head are concat-connected to the obtain multi-modal features. Multi-modal features are classified by using the fully connected network, to obtain the predicted result of mental state. By constructing the mapping relationship between the multi-modal physiological signals and the mental states, the obtained multi-modal physiological features are employed to model the mental perception model, in order to achieve the ultimate goal of knowing people, their faces as well as their minds.

In the above technical scheme, the non-contact physiological signal includes one of the following: heart rate, breathing rate, head vibration, eye movement, blinking rate, line of sight, pupil dilation, lip movement and gait.

In this technical scheme, non-contact physiological signals include one of the following: heart rate, breathing rate, head vibration, eye movement, blinking rate, line of sight, pupil dilation, lip movement, and gait. Head vibration includes frequency, frequency distribution, frequency transformation range, amplitude, amplitude variation range, motion symmetry, and motion period in the horizontal and vertical directions.

In the above-mentioned technical scheme, the mental state includes one or a combination of the following: aggression, stress, anxiety, skepticism, balance, confidence, vitality, regulatory ability, inhibition, sensitivity, depression and happiness.

In this technical scheme, mental states include aggression, stress, anxiety, skepticism, balance, confidence, vitality, regulatory ability, inhibition, sensitivity, depression, and happiness.

In order to achieve the second aspect of the present application, the technical scheme in the second aspect of the present application provides a system for mental state perception, including: an acquisition module, configured for acquiring image sequences with timestamps and millimeter-wave radar raw data with timestamps, where the image sequence comprises a plurality of non-contact physiological signals; a preprocessing module, configured for preprocessing the image sequences and the millimeter-wave radar raw data to obtain head region image sequences, face region image sequences and an original millimeter-wave radar data sequence that are continuous in time series; a head vibration calculation module, configured for analyzing the head region image sequences to obtain head vibration signal feature; a first heart rate calculation module, configured for calculating the face region image sequences by using a remote photovolumetric pulse wave recording method to obtain a first heart rate; a second heart rate calculation module, configured for analyzing the original millimeter-wave radar data sequence to obtain a second heart rate and a breathing rate; a fusion module, configured for fusing the first heart rate, the second heart rate and the breathing rate by using Kalman filtering to obtain a fused heart rate and a fused breathing rate; a facial feature extraction module, configured for performing feature extraction on facial change information in the image sequences by a Transformer-like network to obtain facial motion temporal features; a physiological sequence generation module, configured for corresponding the head vibration signal features, the fused heart rate and the fused breathing rate, and the facial motion temporal features according to timestamps to obtain a corresponding physiological sequence; a prediction module, configured for establishing a non-contact multi-modal mental perception model, and taking the corresponding physiological sequence as the input of the non-contact multi-modal mental perception model to predict and to obtain a predicted result of the mental state.

The system for mental state perception according to the present application comprises an acquisition module, a preprocessing module, a head vibration calculation module, a first heart rate calculation module, a second heart rate calculation module, a fusion module, a physiological sequence generation module and a prediction module, where the acquisition module is configured for acquiring image sequences with timestamps and millimeter-wave radar raw data with timestamps, where the image sequence includes a plurality of non-contact physiological signals. The preprocessing module is configured for preprocessing the image sequences and the millimeter-wave radar raw data to obtain head region image sequences, face region image sequences and an original millimeter-wave radar data sequence that are continuous in time series. The head vibration calculation module is configured for analyzing the head region image sequences to obtain head vibration signal feature. The first heart rate calculation module is configured for calculating the face region image sequences by using a remote photovolumetric pulse wave recording method to obtain a first heart rate. The second heart rate calculation module is configured for analyzing the original millimeter-wave radar data sequence to obtain a second heart rate and a breathing rate. The fusion module, configured for fusing the first heart rate is the second heart rate and the breathing rate by using Kalman filtering to obtain a fused heart rate and a fused breathing rate. The facial feature extraction module is configured for performing feature extraction on facial change information in the image sequences by a Transformer-like network to obtain facial motion temporal features. The physiological sequence generation module is configured for corresponding the head vibration signal features, the fused heart rate and the fused breathing rate, and the facial motion temporal features according to timestamps to obtain a corresponding physiological sequence. The prediction module is configured for establishing a non-contact multi-modal mental perception model, and taking the corresponding physiological sequence as the input of the non-contact multi-modal mental perception model to predict and to obtain a predicted result of the mental state.

By deep learning, combining the Euler motion magnification, it is possible to explore the representation method of the physiological signals of head vibration. Although the intensity of head vibration signal is weak, it has strong periodicity, and it is the signal most significantly related to mental activities. By the fusion of multi-modal physiological signals, the millimeter-wave radar and the rPPG heart rate measurement results are fused, realizing the robust extraction of low signal-to-noise ratio physiological features, and the heart rate and breathing rate measurement results better than single modal are obtained. By progress in the methods for conversion, representation, enhancement of the multi-modal non-contact physiological signals and robust extraction of emotional features, get rid of contact sensing devices, expand application scenarios, and promote the fusion of cross-modal emotional data, so as to improve practical application value in many fields such as man-machine interaction, public safety and medical psychology.

In order to achieve the third aspect of the present application, the technical scheme of the third aspect of the present application provides a system for mental state perception, which includes a memory and a processor, where a program or instruction executable on the processor is stored on the memory, and the processor implements the steps of the method for mental state perception of any one of the schemes of the first aspect when executing the program or instruction. Therefore, it has the technical effect of any of the schemes of the first aspect, and will not be repeatedly described here.

In order to achieve the fourth aspect of the present application, the technical scheme of the fourth aspect of the present application provides a non-transitory computer-readable storage medium on which a computer program is stored. When the program or instruction is executed by a processor, the steps of the method for mental state perception of any one of the schemes of the first aspect are implemented. Therefore, the technical effect of any of the schemes of the first aspect is provided, and will not be repeatedly described here.

Additional aspects and advantages of the present application will become apparent in the description section below or will be understood through the practice of the present application.

### BRIEF DESCRIPTION OF THE DRAWINGS

The aforementioned and/or additional aspects and advantages of the present application will become apparent and understandable from the description of embodiments in conjunction with the following drawings, wherein:

FIG. 1 is a flow diagram schematically showing steps of a method for mental state perception according to an embodiment of the present application;

FIG. 2 is a flow diagram schematically showing steps of a method for mental state perception according to an embodiment of the present application;

FIG. 3 is a flow diagram schematically showing steps of a method for mental state perception according to an embodiment of the present application;

FIG. 4 is a flow diagram schematically showing steps of a method for mental state perception according to an embodiment of the present application;

FIG. 5 is a flow diagram schematically showing steps of a method for mental state perception according to an embodiment of the present application;

FIG. 6 is a block diagram schematically showing a system for mental state perception according to an embodiment of the present application;

FIG. 7 is a block diagram schematically showing a system for mental state perception according to another embodiment of the present application;

FIG. 8 is a flow diagram schematically showing steps of a method for mental state perception according to an embodiment of the present application;

FIG. 9 is a flow diagram schematically showing steps of a method for mental state perception according to an embodiment of the present application;

FIG. 10 is a flow diagram schematically showing steps of a method for mental state perception according to an embodiment of the present application;

FIG. 11 is a flow diagram schematically showing steps of a method for mental state perception according to an embodiment of the present application;

FIG. 12 is a flow diagram schematically showing steps of a method for mental state perception according to an embodiment of the present application;

FIG. 13 is a flow diagram schematically showing steps of a method for mental state perception according to an embodiment of the present application;

FIG. 14 is a flow diagram schematically showing steps of a method for mental state perception according to an embodiment of the present application;

FIG. 15 is a flow diagram schematically showing steps of a method for mental state perception according to an embodiment of the present application; and

FIG. 16 is a flow diagram schematically showing steps of a method for mental state perception according to an embodiment of the present application.

### DETAILED DESCRIPTION OF THE EMBODIMENTS

In order to be able to more clearly understand the above purposes, features and advantages of the present application, the present application will be further described in detail in conjunction with the accompanying drawings and specific embodiments below. It should be noted that embodiments of the present application and features in embodiments may be combined with each other without conflict.

Many specific details are set forth in the following description to facilitate a full understanding of the present application, however, the present application may be implemented in other ways other than those described herein, and therefore the scope of protection of the present application is not limited by the specific embodiments disclosed below.

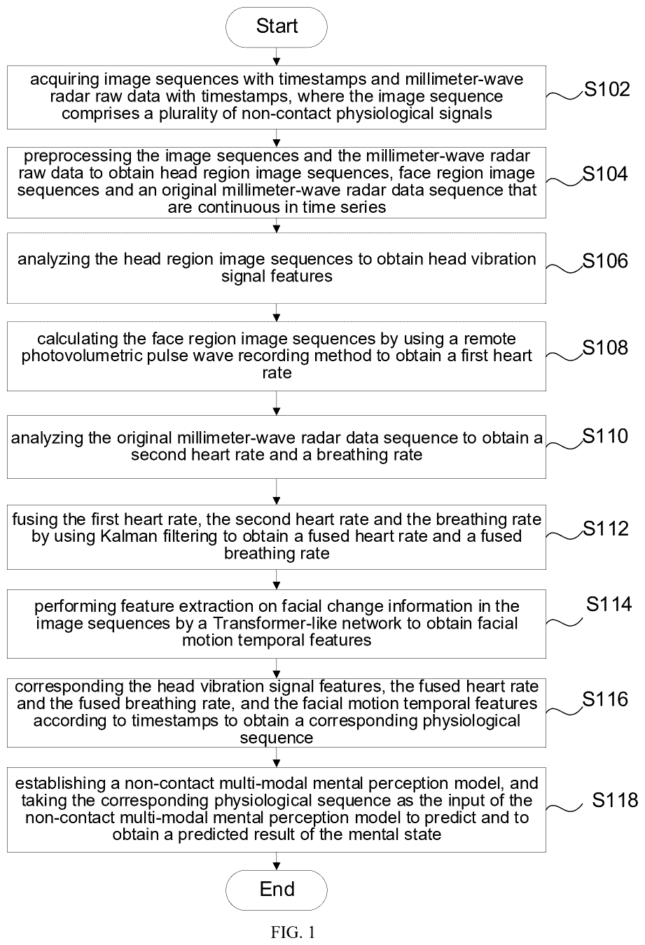

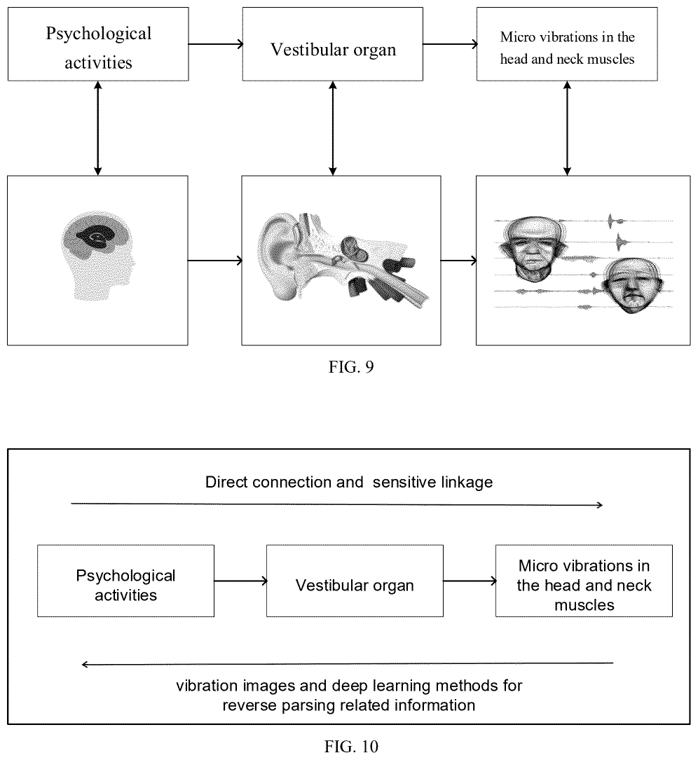

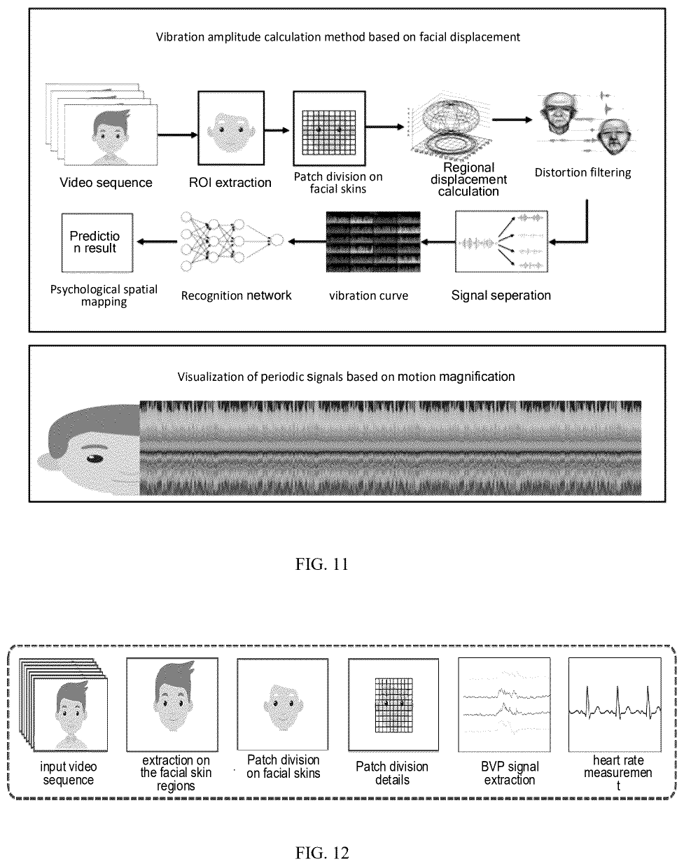

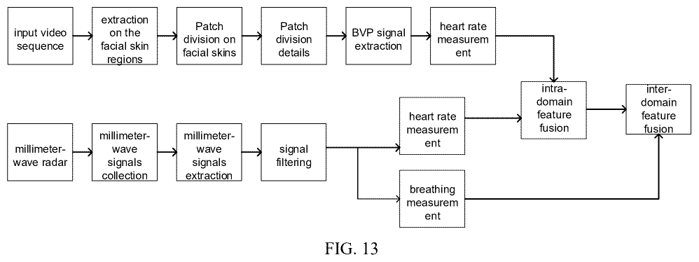

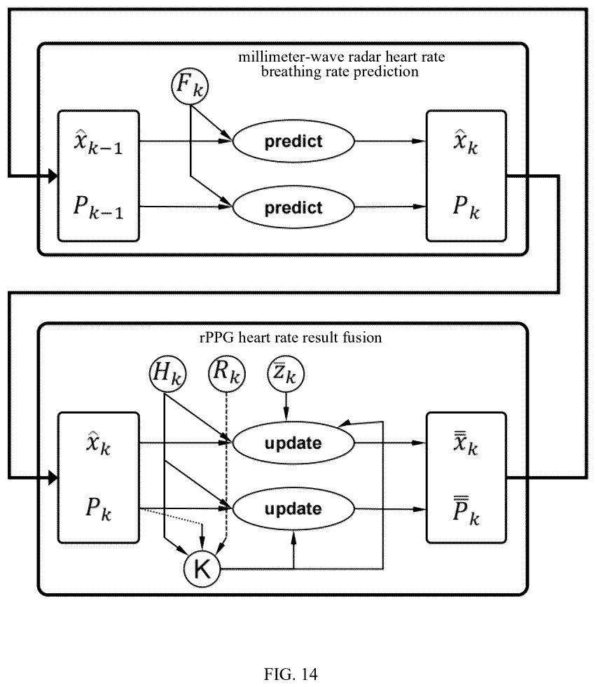

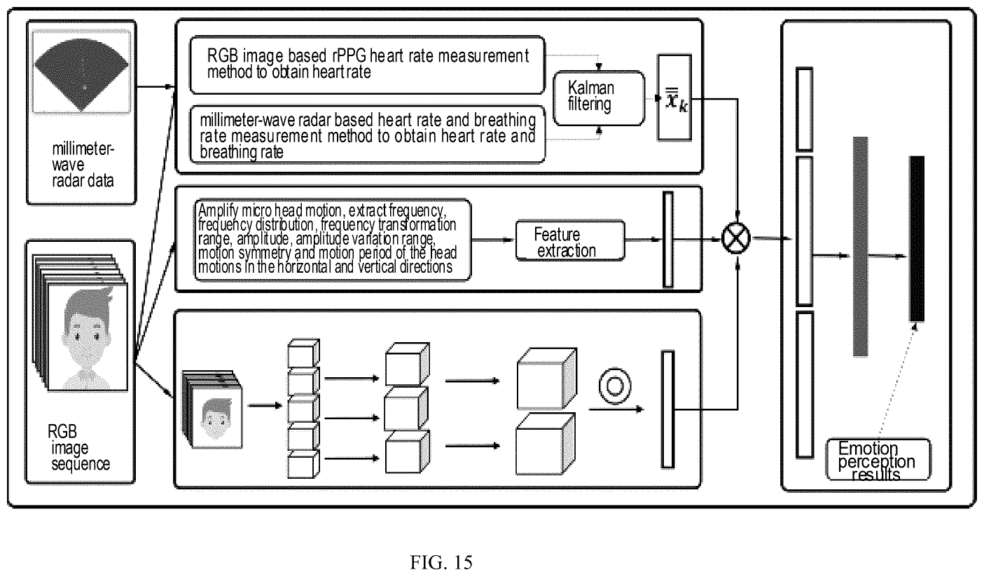

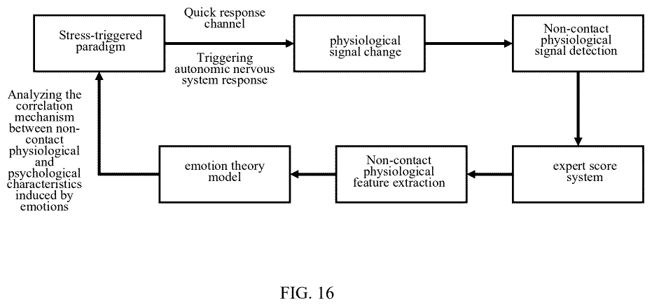

Referring to FIGS. 1 to 16, a method and system for mental state perception, and a readable storage medium according to some embodiments of the present application will be described below.

As shown in FIG. 1, an embodiment of the first aspect of the present application provides a method for mental state perception, including the following steps:
Step S102: acquiring image sequences with timestamps and millimeter-wave radar raw data with timestamps, where the image sequence includes a plurality of non-contact physiological signals;
Step S104: preprocessing the image sequences and the millimeter-wave radar raw data to obtain head region image sequences, face region image sequences and an original millimeter-wave radar data sequence that are continuous in the time series;
Step S106: analyzing the head region image sequences to obtain head vibration signal features;
Step S108: calculating the face region image sequences by using a remote photovolumetric pulse wave recording method to obtain a first heart rate;
Step S110: analyzing the original millimeter-wave radar data sequence to obtain a second heart rate and a breathing rate;
Step S112: fusing the first heart rate, the second heart rate and the breathing rate by using Kalman filtering to obtain a fused heart rate and a fused breathing rate;
Step S114: performing feature extraction on the facial change information in the image sequences by a Transformer-like network to obtain the facial motion temporal feature;
Step S116: corresponding the head vibration signal features, the fused heart rate and the fused breathing rate, and the facial motion temporal features according to timestamps to obtain a corresponding physiological sequence;
Step S118: establishing a non-contact multi-modal mental perception model, and taking the corresponding physiological sequence as the input of the non-contact multi-modal mental perception model to predict and to obtain a predicted result of the mental state.

According to the method for mental state perception provided by the embodiment, first, the image sequences with timestamps and millimeter-wave radar original data with timestamps are acquired, the image sequence and the millimeter-wave radar raw data are preprocessed to obtain the head region image sequences, the face region image sequences and the original millimeter-wave radar data sequence that are continuous in the time series. Then, the head region image sequences is analyzed to obtain the head vibration signal features. The face region image sequences is calculated by using the remote photovolumetric pulse wave recording method so that the first heart rate can be obtained. The original millimeter-wave radar data sequence is analyzed so that the second heart rate and the breathing rate can be obtained. The first heart rate, the second heart rate and the breathing rate are combined so that they can be fused to obtain more precise heart rate and breathing rate. Feature extraction is performed on the facial change information in the image sequences by a Transformer-like network to obtain the facial motion temporal features. The head vibration signal feature, the fused heart rate, the fused breathing rate, and facial motion temporal features are corresponded according to timestamps, taking the corresponding physiological sequence as the input of the non-contact multi-modal mental perception model, for prediction by the non-contact multi-modal mental perception model, to obtain the mental state of an individual being measured.

By deep learning, it is possible to explore the representation method of the physiological signals of head vibration. Although the intensity of head vibration signal is weak, it has strong periodicity, which is the signal most significantly related to mental activities. By the fusion of multi-modal physiological signals, the millimeter-wave radar and the rPPG heart rate measurement results are fused, realizing the robust extraction of low signal-to-noise ratio physiological features, and the heart rate and breathing rate measurement results better than single modal are obtained. By progress in the methods for conversion, representation, enhancement of the multi-modal non-contact physiological signals and robust extraction of emotional features, get rid of contact sensing devices, expand application scenarios, and promote the fusion of cross-modal emotional data, so as to improve practical application value in many fields such as man-machine interaction, public safety and medical psychology.

The head vibration signal is strongly related to mental states, and is one of the signals which most significantly related to mental activities. The principle is: the vertical balance of the human head is controlled by the vestibular system, and individual mental activities will act on the vestibular organs through the cerebral cortex, which in turn affects the vertical balance of the head. This function is called vestibular reflex function. Vestibular organ reflex is an uncontrollable autologous primary vibration, which is not controlled by individual thinking and consciousness, so head vibration is a real reflection of individual mental state. The vestibular reflex function provides a direct connection and sensitive linkage between mental activities and head vibration. By using Euler motion magnification to display subtle head vibrations, and performing reverse parsing of head vibrations by artificial intelligence, the individual's mental and physiological state can be sensed accurately, quickly and unconsciously.

In the above-described embodiment, by using a millimeter-wave radar to detect the fluctuations in the position of the chest cavity caused by human life activities, the second heart rate and the breathing rate are obtained. Specifically, in the millimeter-wave radar, a frame is captured every 50 ms, with each frame consisting of a set of measurements. By accumulating data from N frames, the phase variations over time can be obtained, where the phase variations reflect changes (produced by breathing and heartbeat during physiological activities) in the surface amplitude of the individual being measured. Based on the curve of surface amplitude variations described, selecting an appropriate sliding window, by using 512 frames of data for estimation, that is a sliding window of 25.6 seconds, apply correlation filtering to the phase information.

The phase information is filtered by using two sets of bandpass filters with different cutoff frequencies to filter out the waveform signals of breathing and heartbeat. The filtered signals are then analyzed by using methods such as FFT or peak counting to obtain the second heart rate and breathing rate of the individual being measured.

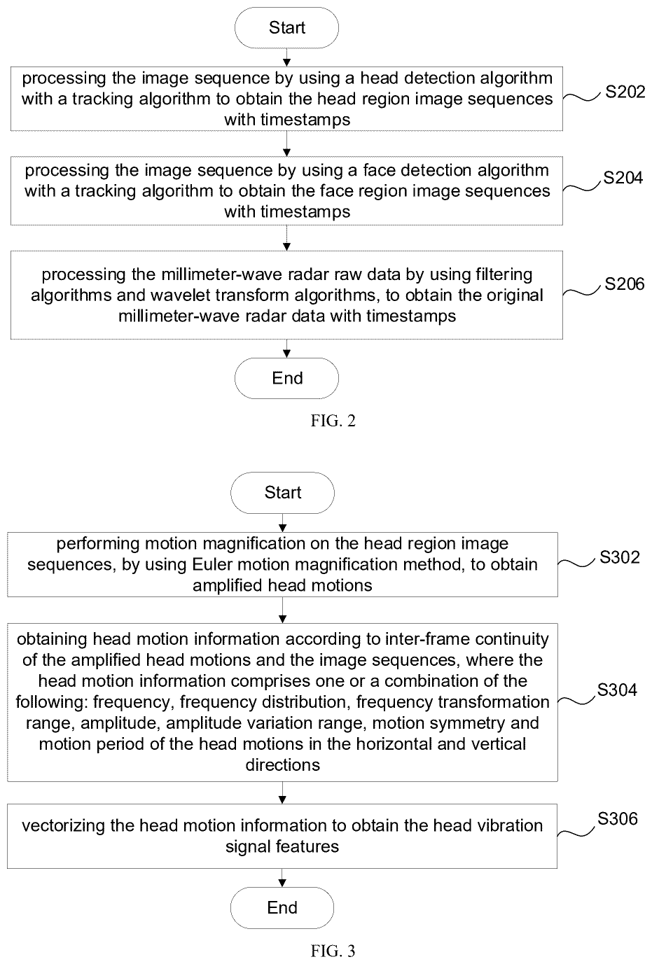

As shown in FIG. 2, according to the method for mental state perception proposed in one embodiment of the present application, the image sequence and the millimeter-wave radar raw data are preprocessed to obtain the head region image sequences, the face region image sequences and the original millimeter-wave radar data sequence that are continuous in time series, the method includes the following steps:
Step S202: processing the image sequence by using a head detection algorithm with a tracking algorithm to obtain head region image sequences with timestamps;
Step S204: processing the image sequence by using a face detection algorithm with a tracking algorithm to obtain face region image sequences with timestamps;
Step S206: performing filtering processing on the millimeter-wave radar raw data by using filtering algorithms and wavelet transform algorithms, to obtain the original millimeter-wave radar data with timestamps.

In this embodiment, the image sequence and the millimeter-wave radar raw data are preprocessed to obtain the head region image sequences, the face region image sequences and the original millimeter-wave radar data sequence that are continuous in the time series. Specifically, existing head detection algorithms are used to process the image sequences, the head region corresponding to each frame of image is cropped, and stored as head region image sequences with timestamp information. Existing face detection algorithms are used to process the image sequences, and the face region corresponding to each frame of the image is cropped, and stored as face region image sequences with timestamp information. The millimeter-wave radar raw data is processed by using filtering algorithms, and the results of processing are stored as the original millimeter-wave radar data sequence with timestamp information.

As shown in FIG. 3, according to the method for mental state perception proposed in one embodiment of the present application, analyzing the head region image sequences to obtain head vibration signal features specifically includes the following steps:
Step S302: performing motion magnification on the head region image sequences, by using Euler motion magnification method, to obtain amplified head motions;
Step S304: obtaining head motion information according to inter-frame continuity of the amplified head motions and the image sequences, where the head motion information comprises one or a combination of the following: frequency, frequency distribution, frequency transformation range, amplitude, amplitude variation range, motion symmetry and motion period of the head motions in the horizontal and vertical directions;
Step S306: vectorizing the head motion information to obtain the head vibration signal features.

In this embodiment, the head region image sequences is analyzed to obtain the head vibration signal features, specifically including, performing motion magnification on the head region image sequences, by using Euler motion magnification method, to obtain amplified head motions; obtaining head motion information according to inter-frame continuity of the amplified head motions and the image sequences; vectorizing the head motion information to obtain the head vibration signal features; where the head motion information comprises one or a combination of the following: frequency, frequency distribution, frequency transformation range, amplitude, amplitude variation range, motion symmetry and motion period of the head motions in the horizontal and vertical directions.

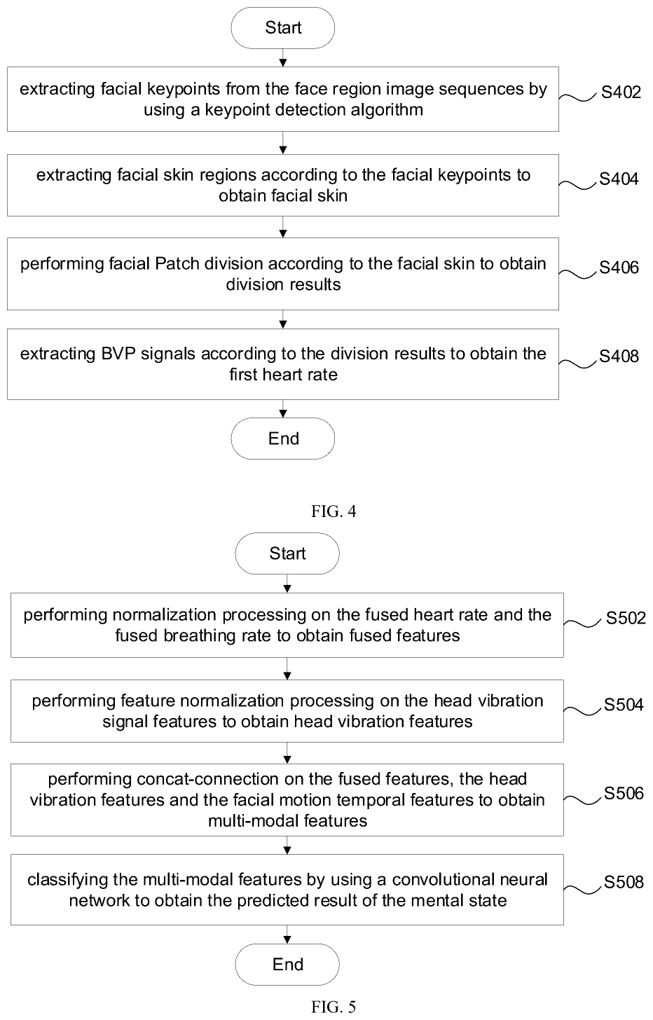

As shown in FIG. 4, according to the method for mental state perception proposed in one embodiment of the present application, calculating the face region image sequences by using the remote photovolumetric pulse wave recording method to obtain the first heart rate includes the following steps:
Step S402: extracting facial keypoints from the face region image sequences by using a keypoint detection algorithm;
Step S404: extracting facial skin regions according to the facial keypoints to obtain facial skin;
Step S406: performing facial Patch division according to the facial skin, to obtain division results;
Step S408: extracting BVP signals according to the division results to obtain the first heart rate.

In the embodiment, calculating the face region image sequences by using the remote photovolumetric pulse wave recording method to obtain the first heart rate specifically includes the following steps: according to the face region image sequences, the facial keypoints are extracted by using a keypoint detection algorithm, and the facial skin regions are extracted according to the extracted keypoints (this process can avoid the interference from a complex background), and then facial Patch division is performed by using the positions of the keypoints. The facial Patch division can avoid the problem of excessive noise of the measurement signal caused by uneven illumination, and the BVP signals are extracted, and the heart rate information of the individual being measured can be finally obtained.

In the above embodiments, the formula for the Kalman filter is:

$${\hat{x}}_{k} = [ \begin{matrix}Heart Rate \\ Breathing Rate\end{matrix} ] ; \\ {P}_{k} = [ \begin{matrix}{Cov}_{hh} & {Cov}_{hb} \\ {Cov}_{bh} & {Cov}_{bb}\end{matrix} ] ; \\ {\hat{x}}_{k} = {F}_{k} {\hat{x}}_{k - 1} ; \\ {P}_{k} = {F}_{k} {P}_{k - 1} {F}_{k}^{T} ; \\ K = {H}_{k} {P}_{k} {{H}_{k}^{T} ( {H}_{k} {P}_{k} {H}_{k}^{T} + {R}_{k} )}^{- 1} ; \\ {{x}^{=}}_{k} = {\hat{x}}_{k} + K ( {\overline{z}}_{k} - {H}_{k} {\hat{x}}_{k} ) ; \\ {{P}^{=}}_{k} = {P}_{k} - {KH}_{k} {P}_{k} ;$$

Among them, \hat{x}ₖ is the millimeter-wave radar measurement value of heart rate and breathing rate at time k, Pₖ is a covariance matrix of heart rate and breathing rate, Covₕₕ represents the covariance of the heart rate and breathing rate in \hat{x}ₖ. Fₖ is the state transition matrix from k−1 to k, Hₖ is the result of rPPG heart rate measurement at time k, Rₖ represents the variance of uncertainty in heart rate measurement, z̅ₖ is the average value of Rₖ, x̅ₖ is the fused heart rate, P̅ₖ is the fused breathing rate, and K is the Kalman filtering gain. Kalman filtering is used to fuse the first heart rate, the second heart rate and the breathing rate. Kalman filtering, based on Bayesian estimation theory and considering the covariance between rPPG and mmWave, assigns larger weights to items with small errors and smaller weights to items with large errors, so as to minimize the error of the predicted result.

As shown in FIG. 5, according to the method for mental state perception proposed in one embodiment of the present application, establishing the non-contact multi-modal mental perception model, and taking the corresponding physiological sequence as the input of the non-contact multi-modal mental perception model to predict and to obtain the predicted result of the mental state, specifically includes the following steps:
Step S502: performing normalization processing on the fused heart rate and the fused breathing rate to obtain fused features;
Step S504: performing feature normalization processing on the head vibration signal features to obtain head vibration features;
Step S506: performing concat-connection on the fused features, the head vibration features and the facial motion temporal features to obtain multi-modal features;
Step S508: classifying the multi-modal features by using a convolutional neural network to obtain the predicted result of the mental state.

In this embodiment, the non-contact multi-modal mental perception model is established, and a corresponding physiological sequence is used as the input of the non-contact multi-modal mental perception model for prediction, so as to obtain the predicted result of the mental state, specifically, the fused heart rate and the fused breathing rate are normalized to obtain fused features. Temporal characteristics of the head vibration are extracted to obtain the features of head vibration. Features are extracted from the temporal information of facial motions in expression and head by using an MViT2 network, to obtain features of facial motion in expression and head. The fused features, the head vibration features, the features of facial motion in expression and head are concat-connected to the obtain multi-modal features. Multi-modal features are classified by using the fully connected network, to obtain the predicted result of mental state. By constructing the mapping relationship between the multi-modal physiological signals and the mental states, the obtained multi-modal physiological features are employed to model the mental perception model, in order to achieve the ultimate goal of knowing people, their faces as well as their minds.

In some embodiments, non-contact physiological signals include one of the following: heart rate, breathing rate, head vibration, eye movement, blinking rate, line of sight, pupil dilation, lip movement, and gait. Head vibration includes frequency, frequency distribution, frequency transformation range, amplitude, amplitude variation range, motion symmetry, and motion period in the horizontal and vertical directions.

In some embodiments, mental states include aggression, stress, anxiety, skepticism, balance, confidence, vitality, regulatory ability, inhibition, sensitivity, depression, and happiness.

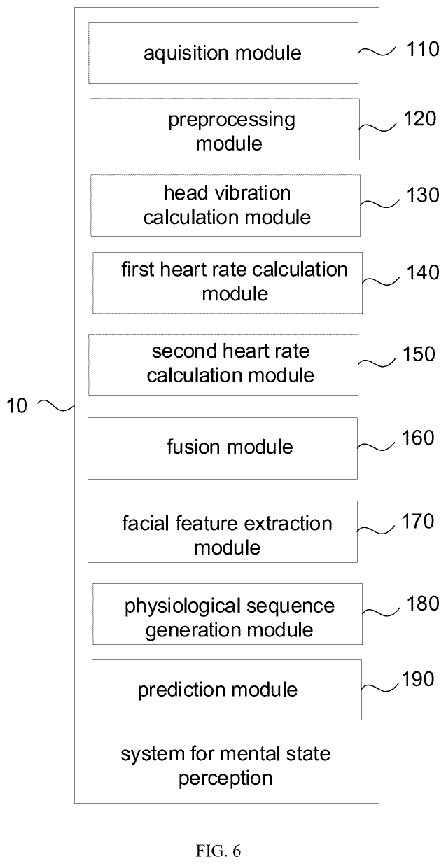

As shown in FIG. 6, an embodiment of a second aspect of the present application provides a system for mental state perception 10, including: an acquisition module 110, configured for acquiring image sequences with timestamps and millimeter-wave radar raw data with timestamps, where the image sequence includes a plurality of non-contact physiological signals; a preprocessing module 120, configured for preprocessing the image sequences and the millimeter-wave radar raw data to obtain head region image sequences, face region image sequences and an original millimeter-wave radar data sequence that are continuous in time series; a head vibration calculation module 130 configured for analyzing the head region image sequences to obtain head vibration signal feature; a first heart rate calculation module 140 configured for calculating the face region image sequences by using a remote photovolumetric pulse wave recording method to obtain a first heart rate; a second heart rate calculation module 150 configured for analyzing the original millimeter-wave radar data sequence to obtain a second heart rate and a breathing rate; a fusion module 160 configured for fusing the first heart rate, the second heart rate and the breathing rate by using Kalman filtering to obtain a fused heart rate and a fused breathing rate; a facial feature extraction module 170 configured for performing feature extraction on facial change information in the image sequences by a Transformer-like network to obtain facial motion temporal features; a physiological sequence generation module 180, configured for corresponding the head vibration signal features, the fused heart rate and the fused breathing rate, and the facial motion temporal features according to timestamps to obtain a corresponding physiological sequence; a prediction module 190, configured for establishing a non-contact multi-modal mental perception model, and taking the corresponding physiological sequence as the input of the non-contact multi-modal mental perception model to predict and to obtain a predicted result of the mental state.

The system for mental state perception 10 according to the embodiment includes an acquisition module 110, a preprocessing module 120, a head vibration calculation module 130, a first heart rate calculation module 140, a second heart rate calculation module 150, a fusion module 160, a facial feature extraction module 170, a physiological sequence generation module 180, and a prediction module 190. The acquisition module 110 is configured for acquiring image sequences with timestamps and millimeter-wave radar raw data with timestamps, where the image sequence includes a plurality of non-contact physiological signals. The preprocessing module 120 is configured for preprocessing the image sequences and the millimeter-wave radar raw data to obtain head region image sequences, face region image sequences and an original millimeter-wave radar data sequence that are continuous in the time series. The head vibration calculation module 130 is configured for analyzing the head region image sequences to obtain head vibration signal features. The first heart rate calculation module 140 is configured for calculating the face region image sequences by using a remote photovolumetric pulse wave recording method to obtain a first heart rate. The second heart rate calculation module 150 is configured for analyzing the original millimeter-wave radar data sequence to obtain a second heart rate and a breathing rate. The fusion module 160 is configured for fusing the first heart rate, the second heart rate and the breathing rate by using Kalman filtering to obtain a fused heart rate and a fused breathing rate. The facial feature extraction module 170 is configured for performing feature extraction on facial change information in the image sequences by a Transformer-like network to obtain facial motion temporal features. The physiological sequence generation module 180 is configured for corresponding the head vibration signal features, the fused heart rate and the fused breathing rate, and the facial motion temporal features according to timestamps to obtain a corresponding physiological sequence. The prediction module 190 is configured for establishing a non-contact multi-modal mental perception model, and taking the corresponding physiological sequence as the input of the non-contact multi-modal mental perception model to predict and to obtain a predicted result of the mental state.

By deep learning, combining the Euler motion magnification, it is possible to explore the representation method of the physiological signals of head vibration. Although the intensity of head vibration signal is weak, it has strong periodicity, and it is the signal most significantly related to mental activities. By the fusion of multi-modal physiological signals, the millimeter-wave radar and the rPPG heart rate measurement results are fused, realizing the robust extraction of low signal-to-noise ratio physiological features, and the heart rate and breathing rate measurement results better than single modal are obtained. By progress in the methods for conversion, representation, enhancement of the multi-modal non-contact physiological signals and robust extraction of emotional features, get rid of contact sensing devices, expand application scenarios, and promote the fusion of cross-modal emotional data, so as to improve practical application value in many fields such as man-machine interaction, public safety and medical psychology.

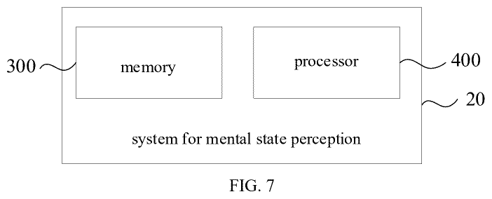

As shown in FIG. 7, a third aspect of the embodiment of the present application provides a system for mental state perception 20, which includes a memory 300 and a processor 400, where a program or instruction executable on the processor 400 is stored on the memory 300, and the processor 400 implements the steps of the method for mental state perception of any one of the embodiments of the first aspect when executing the program or instruction. Therefore, it has the technical effect of any of the embodiments of the first aspect, and will not be repeatedly described here.

The embodiment of a fourth aspect of the present application provides a non-transitory computer-readable storage medium on which a computer program is stored. When the program or instruction is executed by a processor, the steps of the method for mental state perception of any one of the embodiments of the first aspect are implemented. Therefore, the technical effect of any of the embodiments of the first aspect is provided, and will not be repeatedly described here.

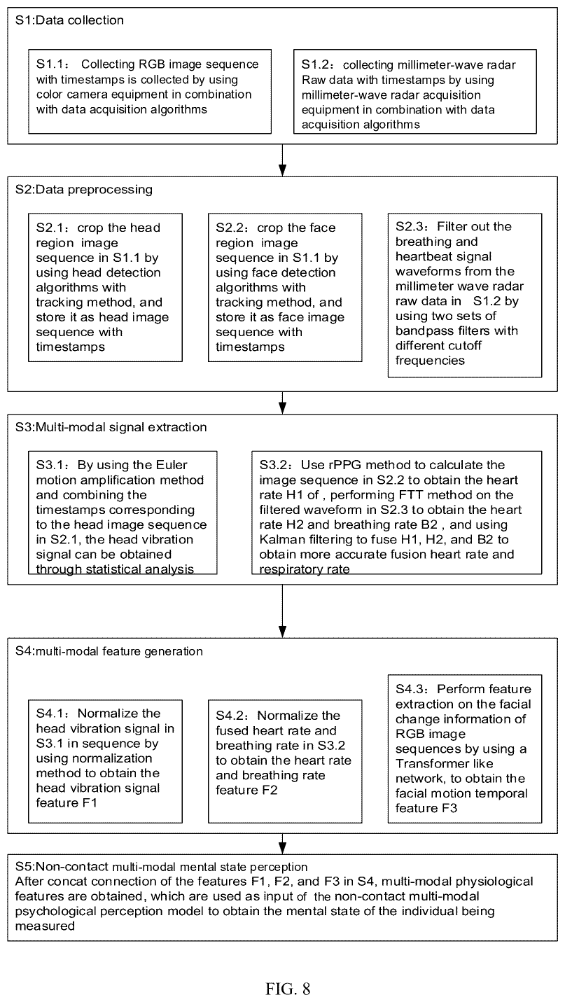

As shown in FIGS. 8 to 16, the method for mental state perception according to one embodiment provided in the present application includes the following steps:
Step S1 includes two sub-steps S1.1 and S1.2. In step S1.1, a sequence of RGB images with timestamps is collected and stored by using an RGB camera. In step S1.2, the millimeter-wave radar equipment is used to collect and store the millimeter-wave radar Raw data sequence with timestamps.
Step S2 includes three sub-steps S2.1, S2.2 and S2.3. In step S2.1, the image sequences collected in step S1.1 is processed by using an existing head detection algorithm, where the head region corresponding to each frame of the image is cropped, and stored as the image sequences with time stamp information. In step S2.2, the image sequences collected in step S1.1 is processed by using an existing face detection algorithm, where the face region corresponding to each frame of the image is cropped, and stored as the image sequence with time stamp information. In step S2.3, a filtering algorithm is used to process the millimeter-wave radar Raw data sequence collected in S1.2, and the processed result is stored as a millimeter-wave radar data sequence with time stamp information.
Step S3 includes two sub-steps S3.1 and S3.2. In Step S3.1, the vibration frequency and vibration amplitude of the head region are calculated by using the Euler motion magnification method and combining the timestamps corresponding to the image sequences. In step S3.2, the corresponding heart rate H1 can be obtained by calculating the image sequences in S2.2 using the rPPG method. By analyzing the filtering results in S2.3, the heart rate H2 and breathing rate B2 can be calculated. Combining H1, H2 and B2, the fusion thereof can be more accurate heart rate and breathing rate.

In step S4, the results in S3.1 and S3.2 are corresponded according to the time stamp, and the corresponded physiological sequence (head vibration features, heart rate, breathing rate) is used as the input of the non-contact multi-modal mental perception model, and the multi-modal mental perception model predicts, and then obtains the mental state of the individual being measured.

This embodiment mainly focuses on heart rate, breathing rate, and head vibration. When an individual is in an anxious or manic state, the envelope of the peripheral blood volume pulse waveform will contract, the body transfers blood from the limbs to important organs and working muscles to prepare for the action response (i.e. “fight or flight” response), causing an imbalance in the body's homeostasis system, accompanied by a series of non-specific physiological responses, mainly manifested as the joint activation of the ANS (Autonomous Nervous System) and the hypothalamus-pituitary-vestibular organ-adrenal axis (Hypothalamic Pituitary Adrenal, HPA). Therefore, by observing heart rate, breathing rate, and head vibrations closely related to the vestibule, the long-term mental status of the individual can be obtained, such as happiness, anxiety, mania, self-confidence, stability, etc.

Existing physiological perception systems face the bottleneck that contact sensing restricts application scenarios and introduces additional emotional interference. The purpose of this application is to study a physiological signal representation method for non-contact multi-modal mental perception, and achieve an accurate and usable non-contact emotional perception system. In a specific implementation, emotional psychology is used as the theoretical guidance, biomedical engineering is used as the methodological basis, and cutting-edge computer science research is used as the key technical means to conduct interdisciplinary research, to get rid of contact sensing devices, expand application scenarios, and promote cross-modal emotional data fusion, by the innovative method of multi-modal non-contact physiological signal conversion, representation, enhancement and robust extraction of emotional features, in order to have practical application value in multiple fields such as human-computer interaction, public safety, and medical psychology.

The theoretical basis of vibration imaging: individual mental activities can be fed back to the vestibular organ. The vestibular organ refers to the three parts (semicircular canal, elliptical capsule and balloon) in the inner ear labyrinth in addition to the cochlea. It is a sensor of the human body's own movement state and head position in space, which controls balance, coordination, muscle tension, and so on. The vertical balance of the human head is controlled by the vestibular system, known as vestibular reflex function. The uncontrollable spontaneous primary vibrations reflected by the vestibular organs can be used to measure an individual's mental state. This is also the technical starting point of vibration imaging methods.

TABLE 1

| | | Condition | | |
|---|---|---|---|---|
| | Degree of | for | Cost for | |
| Techniques | correlation | collection | processing | Main disadvantages |
| micro expression | High | Easy | Low | able to recognize surface emotions but |
| recognition | | | | weak in recognition of deep emotions |
| speech | High | Hard | Low | requiring “cooperation” |
| recognition | | | | |
| gait recognition | Medium | Hard | Low | high difficulty in algorithm |
| | | | | implementation |
| EEG recognition | High | Hard | High | low recognition efficiency |
| thermal imaging | Medium | Easy | High | low accuracy in emotion recognition |
| traditional lie | Medium | Hard | High | low recognition efficiency and low |
| detector | | | | accuracy |
| recognition | | | | |
| vibration image | High | Easy | Low | weak signal strength |
| recognition | | | | |

Table 1 shows a comparison of commonly used techniques for sentiment computing. Compared with other techniques, vibration image recognition has the characteristics of high correlation, easy to collect, and low cost for processing. Its main disadvantage is weak signal strength. Therefore, in this application, existing Euler motion magnification and other techniques are utilized, combined with multi-modal physiological signal fusion, to achieve robust signal extraction under low signal-to-noise ratio conditions.

First, in this application, deep learning is used in combination with Euler motion magnification to explore the representation method of “weak but strong” physiological signals of head vibration. The head vibration signal has weak intensity and strong periodicity, and is the most significant signal associated with psychological activity. Psychological activity acts on the vestibular organ, and the reflex function of the vestibular organ triggers uncontrollable spontaneous primary vibrations in the head and neck muscles. By using vibration images and deep learning methods for reverse parsing, individuals' corresponding psychological activities can be obtained

FIG. 8 shows the process and results of applying the Euler motion magnification method to detection of the head vibrations within a fixed time (such as 2 seconds), and the amplitude of the head motion is determined by the displacement of keypoints on the face.

Second, in this application, multi-modal physiological signal fusion is used, fusing measurement results of millimeter-wave radar and rPPG heart rate, to realize robust extraction of low signal-to-noise ratio physiological features, and obtain better heart rate and breathing rate measurement results than single mode.

The principle of millimeter-wave radar heart rate measurement: two radar waves are transmitted per frame, and the period of each frame is 50 ms.

Waveforms of vital signs are sampled along the “slow time axis”, so the sampling rate of vital signs is equal to the frame rate of the system (that is, within each frame, only one sample is collected, and the phase variations of heart rate and breathing are obtained through N consecutive frames).

The rPPG heart rate measurement process is: perform face detection on the input video sequence, use the keypoint detection algorithm to extract the facial keypoints, and perform extraction on the facial skin regions according to the extracted keypoints (this process can avoid the interference of complex backgrounds), and then use the positions of the keypoints to perform facial Patch division. The facial Patch division can avoid the problem of excessive noise of the measurement signal caused by uneven illumination, and the BVP signals are extracted, and the heart rate information of the individual being measured is finally obtained.

As shown in FIG. 8, Kalman filtering is used to fuse the results (mmWave heart rate, breathing rate rPPG heart rate). Kalman filtering, based on Bayesian estimation theory and considering the covariance between rPPG and mmWave, assigns larger weights to items with small errors and smaller weights to items with large errors, so as to minimize the error of the predicted result.

In the modelling of the system, and at time k, the millimeter-wave radar measurements of heart rate and breathing rate are:

$${\hat{x}}_{k} = [ \begin{matrix}Heart Rate \\ Breathing Rate\end{matrix} ] ;$$

The covariance matrix of heart rate and breathing rate

$${P}_{k} = [ \begin{matrix}{Cov}_{hh} & {Cov}_{hb} \\ {Cov}_{bh} & {Cov}_{bb}\end{matrix} ] ;$$

where Covₕₕ represents the covariance of the heart rate and breathing rate in \hat{x}ₖ;

$${\hat{x}}_{k} = {F}_{k} {\hat{x}}_{k - 1} ; \\ {P}_{k} = {F}_{k} {P}_{k - 1} {F}_{k}^{T} ;$$

where Fₖ is the state transition matrix from k−1 to k,

Hₖ is defined as the result of rPPG heart rate measurement at time k. Rₖ represents the variance of uncertainty in heart rate measurement, z̅ₖ is the average value of Rₖ. The initial value of this value is set to 0, and it can be calculated by the iteration of the Kalman filter later. z̅ₖ is the average value of Rₖ.

Accurate heart rate, breathing rate values, x̅ₖ, P̅ₖ, and the corresponding Kalman filter gain K, obtained b Kalman filtering, are expressed as follows:

$$K = {H}_{k} {P}_{k} {{H}_{k}^{T} ( {H}_{k} {P}_{k} {H}_{k}^{T} + {R}_{k} )}^{- 1} ; \\ {{x}^{=}}_{k} = {\hat{x}}_{k} + K ( {\overline{z}}_{k} - {H}_{k} {\hat{x}}_{k} ) ; \\ {{P}^{=}}_{k} = {P}_{k} - {KH}_{k} {P}_{k} ;$$

Third, in this application, breakthroughs are made against the limitations of knowing people and their faces but not their minds, establish the mapping relationship between multi-modal physiological signals and mental states, establishing a mental perception model based on non-contact physiological signals, to achieve the ultimate goal of knowing people, their faces as well as their minds. After the first point and the second point, the head vibration features amplified by Euler motion and the precise physiological features of heart rate and breathing rate can be obtained. In the third point, the obtained multi-modal physiological features are used to model the mental perception model, in order to achieve the ultimate goal of knowing people, their faces as well as their minds.

As shown in FIG. 6 shows the overall flow chart of multi-modal feature fusion. First, more accurate breathing rate and heart rate x̅ₖ can be obtained by Kalman filtering from the measurement results of rPPG and millimeter-wave radar. Then according to the first point, after amplifying the head motion by using Euler motion magnification, the head motion amplitude and frequency can be obtained, and the head vibration features can be obtained by feature extraction. In order to make better use of head motion information, feature extraction can be performed on the temporal information of facial expression and head motions by using an MViT2 network. The three extracted features are concat-connected to obtain multi-modal features, which are classified by using the full connection layer, and then the final mental state predicted results are obtained.

Specifically, in the method mentioned in the second point, 30 x̅ₖ(3 are measured per second) can be obtained within a certain period of time (10 seconds), serializing them, then two feature vectors with a length of 30 (30 breath values, 30 heart rate values) can be obtained, which are represented as Feature_Breath, Feature_Heart. It should be noted that at this time, the type of the feature value is integer, and it needs to be normalized. The specific processing process is as follows:

$$Feature 1 = \frac{Feature\_Breath}{100} ⊕ \frac{Feature\_Heart}{250}$$

where, the symbol ⊕ indicates that the two features are directly connected, to obtain a normalized Feature1.

For the method mentioned in the first point, by using the head vibration feature extraction model, temporal features within a certain time (10 seconds) can be obtained, and its length is 128, which is expressed as Feature2. For the MViT2 network used for temporal facial expression and head motion feature extraction, features with a length of 128 can be extracted and represented as Feature3.

Feature=Feature₁⊕Feature₂⊕Feature₃;

From this, a multi-modal feature Feature is obtained with a length of 316. After the multi-modal features are fully connected to the network, the final predicted results can be obtained.

The categories of mental states include aggression, stress, anxiety, skepticism, balance, confidence, vitality, regulatory ability, inhibition, sensitivity, depression and happiness.

Last, in this application, a reasonable induction mechanism is designed, to collect mental and physiological data, and analyze the correlation mechanism between non-contact physiological features and mental features under emotional induction. In order to facilitate data acquisition, design a reasonable data acquisition protocol, which can be divided into three aspects: use stroop test and mental arithmetic test to induce stress cognitive pressure, use public interview speech to induce stress tension, and use multimedia data (audio, video, image, text) to induce physiological and mental changes. The final results combine the inducing source and expert scores to obtain the mental label GroundTruth of the individual being measured. As shown in FIG. 8, the innovative point is that first of all, an expert scoring system is introduced in the whole process, and mental experts are used to professionally score the status of the individual being measured, so as to obtain more accurate physiological status. Then, the expert scoring adopts the form of Soft Label, which will score aggression, stress, anxiety, suspicion, balance, self-confidence, vitality, adjustment ability, inhibition, sensitivity, depression, and happiness respectively, and the multi-dimensional mental state of the individual being measured is then obtained. The main reason for adopting Soft Label is that at a certain moment, the individual's mental state is complex and multi-dimensional rather than just a single dimension. Using multi-dimensional representation can more accurately characterize the individual's mental state. Then the more precise correlation mechanism between physiological characteristics and mental characteristics can be obtained by analyzing.

In conclusion, the beneficial effects of the embodiment of the present application are as follows.

Firstly, use deep learning combined with Euler motion magnification to explore the representation method of “weak but strong” physiological signals of head vibration. The head vibration signal has weak intensity but strong periodicity, and is the most significant signal associated with psychological activity.

Secondly, multi-modal physiological signal fusion, fusing measurement results of millimeter-wave radar and rPPG heart rate, to realize robust extraction of low signal-to-noise ratio physiological features, and obtain better heart rate and breathing rate measurement results than single mode.

Thirdly, break through the limitations of knowing people and their faces but not their minds, establish the mapping relationship between multi-modal physiological signals and mental states, establishing a mental perception model based on non-contact physiological signals, to achieve the ultimate goal of knowing people, their faces as well as their minds.

Lastly, design a reasonable induction mechanism, collect mental and physiological data, and analyze the correlation mechanism between non-contact physiological characteristics and mental characteristics under emotional induction.

In this application, the terms “first”, “second”, and “third” are used for descriptive purposes only and should not be construed as indicating or implying relative importance; The term ‘multiple’ refers to two or more, unless otherwise specified. The terms ‘installation’, ‘connection’, ‘connection’, ‘fixation’, etc. should be broadly understood. For example, ‘connection’ can be a fixed connection, a detachable connection, or an integral connection; ‘Connected’ can be directly connected or indirectly connected through an intermediate medium. For ordinary technical personnel in this field, the specific meanings of the above terms in this application can be understood according to the specific situation.

In the description of this application, it should be understood that the terms “up”, “down”, “front”, “back”, etc. indicate orientation or positional relationships based on the orientation or positional relationships shown in the accompanying drawings. This is only for the convenience of describing this application and simplifying the description, and does not indicate or imply that the device or module referred to must have a specific orientation, be constructed and operated in a specific orientation. Therefore, it cannot be understood as a limitation of this application.

In the description of this specification, the terms “one embodiment”, “some embodiments”, “specific embodiments”, etc. refer to the specific features, structures, materials, or characteristics described in conjunction with the embodiments or examples included in at least one embodiment or example of this application. In this manual, the illustrative expressions of the above terms may not necessarily refer to the same embodiments or examples. Moreover, the specific features, structures, materials, or characteristics described can be combined in any one or more embodiments or examples in a suitable manner.

The above are only preferred embodiments of the present application and are not intended to limit the present application. For those skilled in the art, the present application may have various modifications and variations. Any modifications, equivalent substitutions, improvements, etc. made within the spirit and principles of this application shall be included within the scope of protection of this application.

1. A method for mental state perception, comprising:
acquiring image sequences with timestamps and millimeter-wave radar raw data with timestamps, wherein the image sequence comprises a plurality of non-contact physiological signals;
preprocessing the image sequences and the millimeter-wave radar raw data to obtain head region image sequences, face region image sequences and an original millimeter-wave radar data sequence that are continuous in time series;
analyzing the head region image sequences to obtain head vibration signal features;
calculating the face region image sequences by using a remote photovolumetric pulse wave recording method to obtain a first heart rate;
analyzing the original millimeter-wave radar data sequence to obtain a second heart rate and a breathing rate;
fusing the first heart rate, the second heart rate and the breathing rate by using Kalman filtering to obtain a fused heart rate and a fused breathing rate;
performing feature extraction on facial change information in the image sequences by a Transformer-like network to obtain facial motion temporal features;
corresponding the head vibration signal features, the fused heart rate and the fused breathing rate, and the facial motion temporal features according to timestamps to obtain a corresponding physiological sequence; and
establishing a non-contact multi-modal mental perception model, and taking the corresponding physiological sequence as the input of the non-contact multi-modal mental perception model to predict and to obtain a predicted result of the mental state.

2. The method for mental state perception according to claim 1, wherein preprocessing the image sequences and the millimeter-wave radar raw data to obtain the head region image sequences, the face region image sequences and the original millimeter-wave radar data sequence that are continuous in the time series, comprises:
processing the image sequence by using a head detection algorithm with a tracking algorithm to obtain the head region image sequences with timestamps;
processing the image sequence by using a face detection algorithm with a tracking algorithm to obtain the face region image sequences with timestamps; and
processing the millimeter-wave radar raw data by using filtering algorithms and wavelet transform algorithms, to obtain the original millimeter-wave radar data with timestamps.

3. The method for mental state perception according to claim 1, wherein analyzing the head region image sequences to obtain the head vibration signal features comprises:
performing motion magnification on the head region image sequences, by using Euler motion magnification method, to obtain amplified head motions;
obtaining head motion information according to inter-frame continuity of the amplified head motions and the image sequences, wherein the head motion information comprises one or a combination of the following: frequency, frequency distribution, frequency transformation range, amplitude, amplitude variation range, motion symmetry and motion period of the head motions in the horizontal and vertical directions; and
vectorizing the head motion information to obtain the head vibration signal features.

4. The method for mental state perception according to claim 1, wherein calculating the face region image sequences by using the remote photovolumetric pulse wave recording method to obtain the first heart rate comprises:
extracting facial keypoints from the face region image sequences by using a keypoint detection algorithm;
extracting facial skin regions according to the facial keypoints to obtain facial skin;
performing facial Patch division according to the facial skin to obtain division results; and
extracting BVP signals according to the division results to obtain the first heart rate.

5. The method for mental state perception according to claim 1, wherein the formula for the Kalman filter is:
$${\hat{x}}_{k} = [ \begin{matrix}Heart Rate \\ Breathing Rate\end{matrix} ] ; \\ {P}_{k} = [ \begin{matrix}{Cov}_{hh} & {Cov}_{hb} \\ {Cov}_{bh} & {Cov}_{bb}\end{matrix} ] ; \\ {\hat{x}}_{k} = {F}_{k} {\hat{x}}_{k - 1} ; \\ {P}_{k} = {F}_{k} {P}_{k - 1} {F}_{k}^{T} ; \\ K = {H}_{k} {P}_{k} {{H}_{k}^{T} ( {H}_{k} {P}_{k} {H}_{k}^{T} + {R}_{k} )}^{- 1} ; \\ {{x}^{=}}_{k} = {\hat{x}}_{k} + K ( {\overline{z}}_{k} - {H}_{k} {\hat{x}}_{k} ) ; \\ {{P}^{=}}_{k} = {P}_{k} - {KH}_{k} {P}_{k} ;$$
wherein, \hat{x}ₖ is a millimeter-wave radar measurement value of heart rate and breathing rate at time k, Pₖ is a covariance matrix of heart rate and breathing rate, Covₕₕ represents a covariance of the heart rate and breathing rate in \hat{x}ₖ. Fₖ is a state transition matrix from k−1 to k, Hₖ is a result of rPPG heart rate measurement at time k, Rₖ represents a variance of uncertainty in heart rate measurement, z̅ₖ is an average value of Rₖ, x̅ₖ is the fused heart rate, P̅ₖ is the fused breathing rate, and K is Kalman filtering gain.

6. The method for mental state perception according to claim 1, wherein, establishing the non-contact multi-modal mental perception model, and taking the corresponding physiological sequence as the input of the non-contact multi-modal mental perception model to predict and to obtain the predicted result of the mental state comprises:
performing normalization processing on the fused heart rate and the fused breathing rate to obtain fused features;
performing feature normalization processing on the head vibration signal features to obtain head vibration features;
performing concat-connection on the fused features, the head vibration features and the facial motion temporal features to obtain multi-modal features; and
classifying the multi-modal features by using a convolutional neural network to obtain the predicted result of the mental state.

7. The method for mental state perception according to claim 1, wherein, the non-contact physiological signal comprises one of the following: heart rate, breathing rate, head vibration, eye movement, blinking rate, line of sight, pupil dilation, lip movement and gait.

8. The method for mental state perception according to claim 1, wherein, the mental state comprises one or a combination of the following: aggression, stress, anxiety, skepticism, balance, confidence, vitality, regulatory ability, inhibition, sensitivity, depression and happiness.

9. A system for mental state perception, comprising:
an acquisition module, configured for acquiring image sequences with timestamps and millimeter-wave radar raw data with timestamps, wherein the image sequence comprises a plurality of non-contact physiological signals;
a preprocessing module, configured for preprocessing the image sequences and the millimeter-wave radar raw data to obtain head region image sequences, face region image sequences and an original millimeter-wave radar data sequence that are continuous in time series;
a head vibration calculation module, configured for analyzing the head region image sequences to obtain head vibration signal feature;
a first heart rate calculation module, configured for calculating the face region image sequences by using a remote photovolumetric pulse wave recording method to obtain a first heart rate;
a second heart rate calculation module, configured for analyzing the original millimeter-wave radar data sequence to obtain a second heart rate and a breathing rate;
a fusion module, configured for fusing the first heart rate, the second heart rate and the breathing rate by using Kalman filtering to obtain a fused heart rate and a fused breathing rate;
a facial feature extraction module, configured for performing feature extraction on facial change information in the image sequences by a Transformer-like network to obtain facial motion temporal features;
a physiological sequence generation module, configured for corresponding the head vibration signal features, the fused heart rate and the fused breathing rate, and the facial motion temporal features according to timestamps to obtain a corresponding physiological sequence; and
a prediction module, configured for establishing a non-contact multi-modal mental perception model, and taking the corresponding physiological sequence as the input of the non-contact multi-modal mental perception model to predict and to obtain a predicted result of the mental state.

10. A system for mental state perception, comprising:
a memory and a processor, wherein a program or instruction executable on the processor is stored on the memory, and the processor implements the steps of the method for mental state perception according to claim 1 when executing the program or instruction.

11. A non-transitory computer-readable storage medium, wherein a program or instruction is stored on the computer-readable storage medium, the program or the instruction, when executed by the processor, implements the method for mental state perception according to claim 1.
